(19) United States
(12) Patent Application Publication
(10) Pub. No.: US 2021/0379204 A1
(43) Pub. Date: Dec. 9, 2021

(54) METHOD FOR CALCULATING TOLERANCE DOSE OF HUMAN BODY TO SUGAR ALCOHOL AND FUNCTIONAL SUGAR

(71) Applicant: Zhejiang Huakang Pharmaceutical Co., Ltd., Quzhou, Zhejiang (CN)

(72) Inventors: Qile ZUO, Quzhou, Zhejiang (CN); Feifei HAN, Quzhou, Zhejiang (CN); Xuan ZHU, Quzhou, Zhejiang (CN); Mian LI, Quzhou, Zhejiang (CN); Wenyao ZHANG, Quzhou, Zhejiang (CN); Lihua SHI, Quzhou, Zhejiang (CN)

(21) Appl. No.: 17/286,006

(22) PCT Filed: Nov. 22, 2019
(86) PCT No.: PCT/CN2019/120304

§ 371 Date: Apr. 16, 2021

(30) Foreign Application Priority Data
Nov. 23, 2018 (CN) 201811422592.0

(51) Int. Cl. A61K 49/00; G16H 50/30

(52) U.S. Cl. CPC A61K 49/0004; G16H 50/30

(57) ABSTRACT

The present disclosure relates to a method for calculating a tolerated dose of a human body to sugar alcohol and functional sugar. Taking xylitol as an example, the method includes the following steps: selecting 200 g rats as experimental animal objects, and dividing the 200 g rats into six experimental groups and one control group, with each including ten numbered rats; gavaging different doses of xylitol to the rats in the six experimental groups each day; gavaging the same dose of sucrose to the rats in the control group each day; providing enough drinking water and common feed to all the experimental rats each day; checking and making statistics on the quantity of the rats with diarrhea every night; continuing for 14 days; by combining the diarrhea situations of rats on the last day with results of intestinal segment slice maps and diamine oxidase contents of the rats in each group, obtaining a maximum tolerated dose N of the rats in the group to xylitol; and obtaining the tolerated dose of the human body to the xylitol by converting the maximum tolerated dose of the rat based on the conversion formula of drug doses of experimental animal and human body. A more accurate tolerated dose range of the human body to sugar alcohol and functional sugar is obtained through animal experiments.

### FIELD OF THE INVENTION

The present disclosure belongs to the technical field of application of sugar alcohol, and particularly relates to a method for calculating a tolerated dose of a human body to sugar alcohol and functional sugar.

### BACKGROUND OF THE INVENTION

The many benefits of sugar alcohol for a human body are self-evident, but excessive intake also has certain adverse effect on the human body, for example, daily excessive intake of sugar alcohol may cause diarrhea and other situations. Since sugar alcohol enters intestines directly without being decomposed by gastric enzyme, an absorption time is longer than that of glucose in small intestine due to its molecular structure different from sugar, and a large amount of intake in a short time will cause borborygmus and diarrhea. At the same time, some companies have ever declared that the daily intake of xylitol should not exceed 50 g, the daily intake of maltitol should not exceed 100 g, the daily intake of L-arabinose should not exceed 120 g, and the daily intake of sorbitol should not exceed 50 g, etc. However, because no corresponding experimental methods and experimental data prove the above and the dosage requirements are too broad, they have no significant instruction significance for the amount of sugar alcohol added in products.

### SUMMARY OF THE INVENTION

The technical problem to be solved by the present disclosure is to provide a method for calculating a tolerated dose of a human body to sugar alcohol and functional sugar. By this method, a more accurate range of the tolerated dose of the human body to sugar alcohol and functional sugar can be obtained through animal experiments.

The present disclosure is realized by providing a method for calculating a tolerated dose of a human body to sugar alcohol and functional sugar, including the following steps:

at step 1, selecting 200 g rats as experimental animal objects, and dividing the 200 g rats into six experimental groups and a control group, with each group including ten numbered rats; gavaging different doses of xylitol to the rats in the six experimental groups each day, wherein the ten rats in each group are gavaged with the same dose; gavaging the same dose of sucrose or normal saline to the ten rats in the control group each day; providing enough drinking water and common feed to all the experimental rats each day; checking whether there is fecal matter at the anus of each rat in each group every night, wherein the existence of the fecal matter indicates that the rat has diarrhea, and making statistics on the quantity and numbers of rats with diarrhea;

at step 2, continuing the step 1 for 14 days;

at step 3, by combining the diarrhea situations of rats on the last day (day 14) with results of intestinal segment slice maps and diamine oxidase contents of the rats in each group, using a maximum dose group in which all the rats do not have diarrhea to obtain a maximum tolerated dose N of the rats in the group to xylitol;

at step 4, converting the maximum tolerated dose N of the 200 g rats to xylitol according to the following conversion formula of drug doses of experimental animal and human to obtain a tolerated dose R of the human body to xylitol:
70 kg-human-body dose R=maximum tolerated dose N of 200 g rat×K,

wherein K is a conversion factor between a 70 kg human body and a 200 g rat, which is 56.0; the maximum tolerated dose N of the rat to xylitol and the tolerated dose R of the human body to the xylitol are both in the unit of g/d; or,

at step 5, replacing the xylitol in the step 1 with L-arabinose, and repeating the step 1 to the step 4 above to obtain a tolerated dose of a 70 kg human body to L-arabinose; or,

replacing the xylitol in the step 1 with sorbitol, and repeating the step 1 to the step 4 to obtain a tolerated dose of a 70 kg human body to sorbitol.

Compared with the prior art, according to the method for calculating the tolerated dose of the human body to sugar alcohol and functional sugar in the present disclosure, a method for calculating a tolerated dose of a human body to sugar alcohol and functional sugar is established by using the most commonly-used xylitol, L-arabinose and sorbitol as the experimental materials and 200 g Wistar rats in animal experiments as the experimental objects in combination with the dose conversion between a human body and an animal in the animal experiments, so as to obtain a more accurate range of the tolerated dose of the human body to sugar alcohol and functional sugar.

### BRIEF DESCRIPTION OF THE DRAWINGS

FIG. 1 is a statistical diagram of diarrhea situations of rats in experimental groups when a xylitol tolerated dose test is performed for rats according to the present disclosure.

FIG. 2 is a comparison diagram of diarrhea situations of rats in experimental groups on the last day when a xylitol tolerated dose test is performed for rats according to the present disclosure.

FIG. 3 is a statistical diagram of diarrhea situations of rats in experimental groups when an L-arabinose tolerated dose test is performed for rats according to the present disclosure.

FIG. 4 is a comparison diagram of diarrhea situations of rats in experimental groups on the last day when an L-arabinose tolerated dose test is performed for rats according to the present disclosure.

FIG. 5 is a statistical diagram of diarrhea situations of rats in experimental groups when a sorbitol tolerated dose test is performed for rats according to the present disclosure.

FIG. 6 is a comparison diagram of diarrhea situations of rats in experimental groups on the last day when a sorbitol tolerated dose test is performed for rats according to the present disclosure.

### DETAILED DESCRIPTION OF THE EMBODIMENTS

In order to make the technical problems to be solved, technical solutions and advantageous effects of the present disclosure clearer, the present disclosure will be described in detail below with reference to the accompanying drawing and embodiments. It should be understood that the specific embodiments described herein are merely illustrative of the present disclosure and are not intended to limit the disclosure.

A preferred embodiment of a method for calculating a tolerated dose of a human body to sugar alcohol and functional sugar in the present disclosure includes the following steps 1-5.

At step 1, 200 g rats are selected as experimental animal objects, and the 200 g rats are divided into six experimental groups and one control group, with each group including ten numbered rats; and the rats in the six experimental groups (namely, dose (1) experimental group, dose (2) experimental group, dose (3) experimental group, dose (4) experimental group, dose (5) experimental group and dose (6) experimental group) are gavaged with different doses of xylitol each day. The ten rats in each group are gavaged with the same dose. The ten rats in the control group are gavaged with the same dose of sucrose or normal saline each day; enough drinking water and common feed are provided to all the experimental rats each day; whether there is fecal matter at the anus of each rat in each group is checked every night, wherein the existence of the fecal matter indicates that the rat has diarrhea, and statistics are made on the quantity and numbers of rats with diarrhea.

At step 2, the step 1 is continued for 14 days.

At step 3, by combining the diarrhea situations of rats on the last day (day 14) with results of intestinal segment slice maps and diamine oxidase contents of the rats in each group, a maximum dose group in which all the rats do not have diarrhea is used to obtain a maximum tolerated dose N of the rats in the group to xylitol.

The results of the intestinal segment slices and the diamine oxidase of the rats prove that the rats with diarrhea found through observation are subjected to gastrointestinal damage due to their doses exceeding a normal tolerated dose, and the intestinal segment slice maps and the diamine oxidase contents of the rats still having diarrhea on the last day (day 14) are significantly different from those of the rats in the control group and the rats without diarrhea. Therefore, the maximum dose group in which all the rats in this group do not have diarrhea on the last day can be taken as the maximum tolerated dose.

At step 4, the maximum tolerated dose N of the 200 g rats to xylitol is converted according to the following conversion formula of drug doses of experimental animal and human body to obtain a tolerated dose R of the human body to xylitol:
70 kg-human-body dose R=maximum tolerated dose N of 200 g rat×K

wherein K is a conversion factor between a 70 kg human body and a 200 g rat, which is 56.0. The maximum tolerated dose N of the rat to xylitol and the tolerated dose R of the human body to the xylitol are both in the unit of g/d; or,

at step 5, the xylitol in the step 1 is replaced with L-arabinose, and the steps 1-4 above are repeated to obtain a tolerated dose of the 70 kg human body to L-arabinose; or,

the xylitol in the step 1 is replaced with sorbitol, and the steps 1-4 are repeated to obtain a tolerated dose of the 70 kg human body to sorbitol.

In the specific implementation, the maximum tolerated dose of the human body to a certain sugar alcohol or functional sugar recorded in the existing data is used as a reference value firstly. Taking xylitol as an example, the existing companies have declared that the daily intake of xylitol should not exceed 50 g. 50 g of xylitol is set as the reference value, and fluctuation is performed based on the reference value to obtain an estimated value of the experimental range. According to the conversion formula of drug doses of experimental animal and human body, the daily experimental dose of 200 g rats to xylitol is obtained through conversion, the group experiment is performed according to the daily experimental dose and statistics are made on the experimental results each day. The experiment is continued for 14 days. Considering the experimental results and verification results comprehensively, the maximum tolerated dose N of 200 g rats to xylitol is obtained. Then, according to the maximum tolerated dose N, the most appropriate maximum tolerated dose R of the human body to xylitol is obtained by conversion based on the conversion formula of drug doses of experimental animal and human body.

Similarly, the maximum tolerated doses of the human body to L-arabinose and sorbitol can be obtained.

Specifically, 50 g of xylitol was set as the reference value, and estimated values of the experimental range of the human body to xylitol were set to 20 g/d, 30 g/d, 40 g/d, 50 g/d, 60 g/d and 70 g/d (six groups in total) according to the reference value. According to the conversion formula of drug doses of experimental animal and human body, the daily experimental dose of xylitol of 200 g rats was respectively obtained as 0.36 g/d, 0.54 g/d, 0.71 g/d, 0.89 g/d, 1.07 g/d and 1.25 g/d (six groups in total), and then experiments were performed respectively.

Similarly, estimated values of the human body to L-arabinose were respectively set to 70 g/d, 80 g/d, 90 g/d, 100 g/d, 110 g/d and 120 g/d (six groups in total) as the experimental range. According to the conversion formula of drug doses of experimental animal and human body, the daily experimental dose of L-arabinose of 200 g rats was respectively obtained as 1.25 g/d, 1.43 g/d, 1.61 g/d, 1.79 g/d, 1.96 g/d and 2.14 g/d (six groups in total), and then experiments were performed respectively.

Similarly, estimated values of the human body to sorbitol were respectively set to 5 g/d, 10 g/d, 15 g/d, 20 g/d, 25 g/d and 30 g/d (six groups in total) as the experimental range. According to the conversion formula of drug doses of experimental animal and human body, the daily experimental dose of sorbitol of 200 g rats was respectively obtained as 0.09 g/d, 0.18 g/d, 0.27 g/d, 0.36 g/d, 0.45 g/d and 0.54 g/d (six groups in total), and then experiments were performed respectively.

The experimental dose of 200 g rats in the control group was 0.5 g/d sucrose solution.

FIG. 1 to FIG. 6 are the statistics of diarrhea situations of 200 g rats for sugar alcohol and functional sugar during an experimental period. For each sugar alcohol or functional sugar, the experimental rats theoretically have a maximum sugar alcohol or functional sugar tolerated dose threshold, so the following three situations may occur during feeding:

(1) no diarrhea occurred during the experimental period;

(2) the diarrhea occurred at the beginning, then improved until the diarrhea disappeared (rats had tolerance adaptivity);

(3) diarrhea existed throughout the experimental period.

Therefore, the maximum tolerated dose is divided according to the above: situation (1) and situation (2) are regarded as within the tolerable range, situation (3) is regarded as beyond the tolerable range, and the maximum dose of situation (2) is considered to be the maximum tolerated dose of the sugar alcohol or functional sugar.

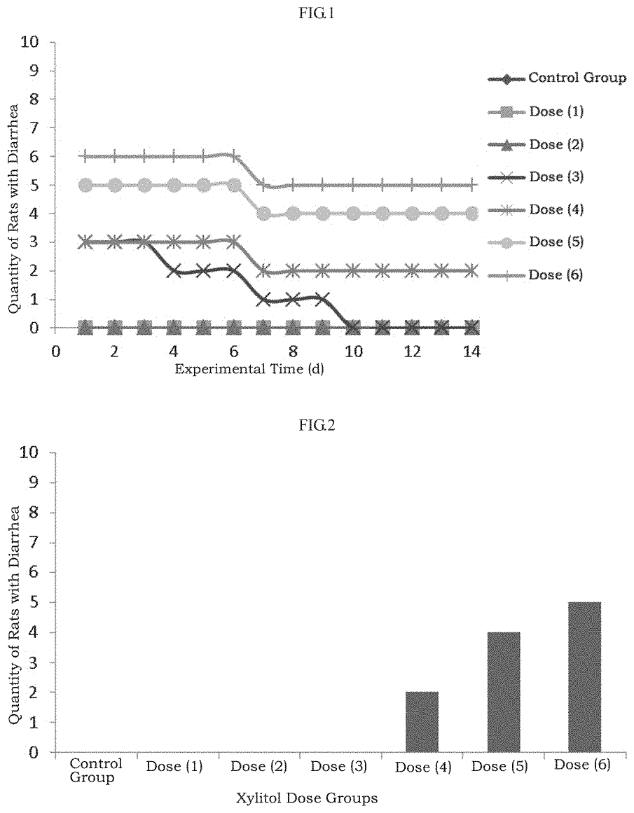

Referring to FIG. 1 and FIG. 2, when the xylitol tolerated dose experiment was performed with the rats, the maximum sugar tolerance dose was dose (3), that is, the dose for gavaging the rats was 0.71 g/d, and the maximum tolerated dose of the human body to xylitol was calculated as 0.71*56.0=40 g/d according to the conversion formula of drug doses of experimental animal and human body. The maximum tolerated dose 40 g of the human body to xylitol obtained by the method of the present disclosure is more specific, more accurate and more well-grounded than the currently-known daily xylitol intake amount of the human body which is preferably no more than 50 g.

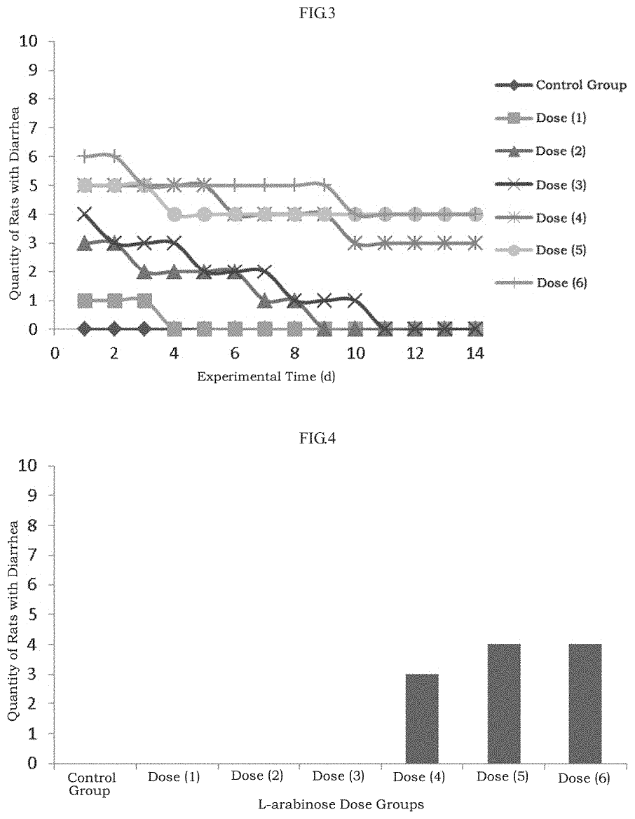

Referring to FIG. 3 and FIG. 4, when the L-arabinose tolerated dose experiment was performed with the rats, the maximum sugar tolerance dose was dose (3), that is, the dose for gavaging the rats was 1.61 g/d, and the maximum tolerated dose of the human body to xylitol was calculated as 1.61*56.0=90 g/d according to the conversion formula of drug dose of experimental animal and human body.

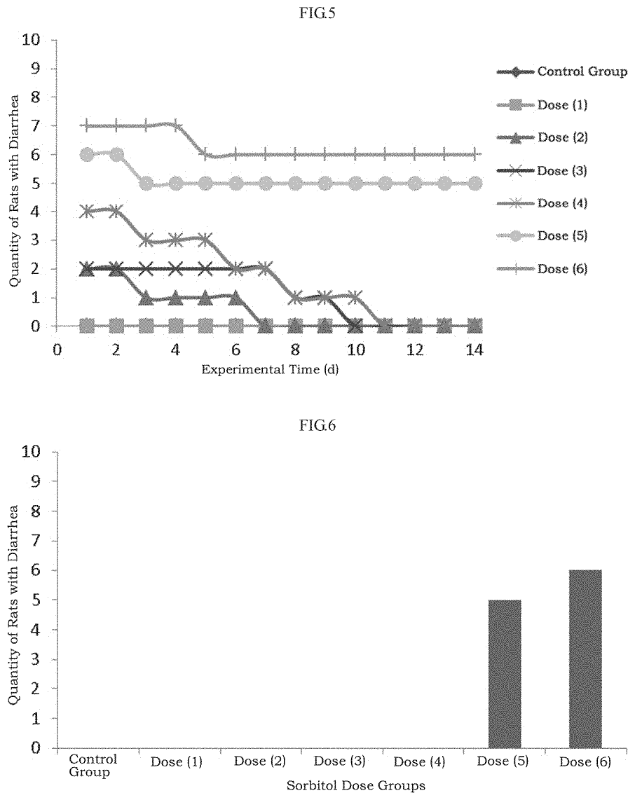

Referring to FIG. 5 and FIG. 6, when the sorbitol tolerated dose experiment was performed with the rats, the maximum sugar tolerance dose was dose (4), that is, the dose for gavaging the rats was 0.36 g/d, and the maximum tolerated dose of the human body to xylitol was calculated as 0.36*56.0=20 g/d according to the conversion formula of drug doses of experimental animal and human body.

The above description is only the preferred embodiments of the disclosure and is not intended to limit the disclosure. Any modifications, equivalent substitutions and improvements made within the spirit and scope of the disclosure should be included within the protection scope of the disclosure.

1. A method for calculating a tolerated dose of a human body to a sugar alcohol or sugar, comprising the following steps:
at step 1, selecting 200 g rats as experimental animal objects, and dividing the 200 g rats into six experimental groups and one control group, with each including ten numbered rats;
gavaging different doses of the sugar alcohol or sugar to the rats in the six experimental groups each day, wherein the ten rats in each group are gavaged with the same dose, and gavaging the same dose of sucrose or saline to the ten rats in the control group each day; providing enough drinking water and common feed to all the experimental rats each day, checking whether there is fecal matter at the anus of each rat in each group every night, wherein the existence of the fecal matter indicates that the rat has diarrhea, and making statistics on the numbers of rats with diarrhea;
at step 2, continuing the step 1 for 14 days;
at step 3, by combining the diarrhea situations of rats on the last day (day 14) with results of intestinal segment slice maps and diamine oxidase contents of the rats in each group, using a maximum dose group in which all the rats do not have diarrhea to obtain a maximum tolerated dose N of the rats in the group to xylitol;
at step 4, converting the maximum tolerated dose N of the 200 g rats to the sugar alcohol or sugar according to the following conversion formula of drug doses of experimental animal and human body to obtain a tolerated dose R of the human body to xylitol:
70 kg-human-body dose R=maximum tolerated dose N of 200 g rat×K
wherein K is a conversion factor between a 70 kg human body and a 200 g rat, which is 56.0; the maximum tolerated dose N of the rat to the sugar alcohol or sugar and the tolerated dose R of the human body to the the sugar alcohol or sugar are both in the unit of g/d.

2. The method of claim 1, wherein the method determines a tolerated dose of a human body to a sugar alcohol.

3. The method of claim 2, wherein the sugar alcohol is xylitol.

4. The method of claim 2, wherein the sugar alcohol is sorbitol.

5. The method of claim 1, wherein the method determines a tolerated dose of a human body to a sugar.

6. The method of claim 5, wherein the sugar is L-arabinose.

7. A method for determining a tolerated dose of a human body to a sugar alcohol or a sugar, comprising the following steps:
gavaging different doses of the sugar alcohol or sugar to rats having a preselected weight in a plurality of experimental groups each day for a predetermined number of days, wherein the rats in each experimental group are fed with the same dose,
determining the numbers of rats in each experimental group with diarrhea;
selecting a maximum dose group in which all the rats do not have diarrhea to obtain a maximum tolerated dose N to the sugar alcohol or sugar; and
converting the maximum tolerated dose N to the sugar alcohol or sugar according to the following conversion formula of drug doses of experimental animal and human body to obtain a tolerated dose R of the human body to the sugar alcohol or sugar:
70 kg-human-body dose R=maximum tolerated dose N of a rat×K
wherein K is a conversion factor between a 70 kg human body and a rat with the preselected weight, and the maximum tolerated dose N of the rat to the sugar alcohol or sugar and the tolerated dose R of the human body to the sugar alcohol or sugar are both in the unit of g/d.

8. The method of claim 7, further comprising gavaging the same dose of sucrose or saline to rats in a control group each day for the predetermined number of days.

9. The method of claim 7, wherein the plurality of experimental groups include six experimental groups.

10. The method of claim 7, wherein each of the plurality of experimental groups includes ten rats.

11. The method of claim 7, wherein the plurality of experimental groups include rats having the same weight.

12. The method of claim 7, wherein the preselected weight is 200 g.

13. The method of claim 7, wherein the gavaging step is performed for 14 days.

14. The method of claim 7, wherein the method determines a tolerated dose of a human body to a sugar alcohol.

15. The method of claim 14, wherein the sugar alcohol is xylitol.

16. The method of claim 14, wherein the sugar alcohol is sorbitol.

17. The method of claim 7, wherein the method determines a tolerated dose of a human body to a sugar.

18. The method of claim 17, wherein the sugar is L-arabinose.
